(19) United States
(12) Patent Application Publication
(10) Pub. No.: US 2026/0106014 A1
(43) Pub. Date: Apr. 16, 2026

(54) DYNAMICALLY GENERATING A TREATMENT PATH FOR EXTRAVASATION OR INFILTRATION

(71) Applicant: IV LEAKS, LLC, Flemington, NJ (US)

(72) Inventors: MICHAEL STRALEY, Flemington, NJ (US); STEPHEN HAYMAN, New York, NY (US); LUIGI BRUNETTI, Scotch Plains, NJ (US); ELLY OH, Wallingford, CT (US); KENDRA STRALEY, Flemington, NJ (US)

(21) Appl. No.: 18/912,434

(22) Filed: Oct. 10, 2024

(51) Int. Cl. G16H 20/17

(52) U.S. Cl. CPC G16H 20/17

(57) ABSTRACT

A treatment path for extravasation or infiltration can be dynamically generated. A system can be configured to allow a clinician to select a drug that was being administered to a patient when a suspected infiltration or extravasation occurred. When the clinician selects a drug, the system can be configured to present various values for each of multiple characteristics applicable to infiltration and extravasation where sets of these values are grouped to define various grades for the infiltration or extravasation. Accordingly, the clinician can select a grade that is associated with a set of values that best represent the characteristics the patient is exhibiting. Based on the selected drug and grade, the system can dynamically generate a treatment path and present the treatment path to the clinician for immediate action. In this way, the infiltration or extravasation can be accurately and immediately treated to thereby minimize harm to the patient.

### CROSS-REFERENCE TO RELATED APPLICATIONS

N/A

### BACKGROUND

When an intravenous (IV) line is used, various complications may arise including infiltration and extravasation, both of which involve the leakage of a fluid out of the vein and into the surrounding tissue. When the leaked fluid is a non-vesicant solution, it is referred to as infiltration. In contrast, when the leaked fluid is a vesicant (i.e., an agent that causes blistering), it is referred to as extravasation.

When infiltration or extravasation occurs, a wide range of treatment options may exist depending on many factors. Therefore, it is difficult for the clinician to determine which treatment option is suitable for any given case. Furthermore, the efficacy of treatment is oftentimes highly dependent on how quickly it can be performed. Yet, the selection of a suitable treatment, particularly with extravasation, oftentimes involves consultations with a specialized doctor and pharmacist who oftentimes must consult literature to arrive at the suitable treatment. Due to these difficulties, the harm to the patient is frequently worse than it otherwise could have been if a treatment path had been immediately and dynamically determined and provided to the clinician.

### BRIEF SUMMARY

Embodiments of the present disclosure are generally directed to methods, systems and computer program products for dynamically generating a treatment path for extravasation or infiltration. A system can be configured to allow a clinician to select a drug that was being administered to a patient when a suspected infiltration or extravasation occurred. When the clinician selects a drug, the system can be configured to present various values for each of multiple characteristics applicable to infiltration and extravasation where sets of these values are grouped to define various grades for the infiltration or extravasation. Accordingly, the clinician can simply select a grade that is associated with a set of values that best represent the characteristics the patient is exhibiting. Based on the selected drug and the selected grade, the system can dynamically generate a treatment path and present the treatment path to the clinician for immediate action. In this way, the infiltration or extravasation can be accurately and immediately treated to thereby minimize harm to the patient.

In some embodiments, a method for dynamically generating a treatment path for infiltration or extravasation is provided. One or more user interfaces can be presented to a clinician. The one or more user interface may allow the clinician to select, from a plurality of drugs, a drug that has infiltrated or extravasated during administration to a patient and to select, from a plurality of grades, a grade of the infiltration or extravasation. An application programming interface (API) server can receive one or more API calls that identify the selected drug and the selected grade. In response to the one or more API calls that identify the selected drug and the selected grade, the API server can query one or more data structures to identify one or more entries that associate the combination of the selected drug and the selected grade with one or more treatment considerations. A treatment path for the infiltration or extravasation can then be presented. The treatment path can include the one or more treatment considerations that are associated with the combination of the selected drug and the selected grade such that the clinician receives the treatment path by selecting the drug and grade.

In some embodiments, presenting the one or more interfaces by which the clinician selects the grade of the infiltration or extravasation comprises presenting a plurality of characteristics of infiltration or extravasation and a plurality of sets of values for the plurality of characteristics where each set of values corresponds with one of the plurality of grades.

In some embodiments, the sets of values for the plurality of characteristics are obtained in response to the clinician's selection of the drug.

In some embodiments, the plurality of characteristics are selected from a group comprising two or more of: pain at the site, skin color, erythema, skin integrity, swelling, catheter flushes, capillary refill, or distal pulses.

In some embodiments, the plurality of characteristics are selected from a group comprising all of: pain at the site, skin color, erythema, skin integrity, swelling, catheter flushes, capillary refill, and distal pulses.

In some embodiments, the plurality of grades include five grades.

In some embodiments, the method includes querying the one or more data structures to identify one or more entries that associate the combination of the selected drug and the selected grade with one or more initial management recommendations. The treatment path can also include the one or more initial management recommendations.

In some embodiments, the method includes querying the one or more data structures to identify one or more entries that associate the combination of the selected drug and the selected grade with one or more post care management recommendations. The treatment path can also include the one or more post care management recommendations.

In some embodiments, the method includes querying the one or more data structures to identify one or more entries that associate the combination of the selected drug and the selected grade with one or more additional recommendations. The treatment path can also include the one or more additional recommendations.

In some embodiments, the one or more additional recommendations can include a surgical consult recommendation or a reassessment criteria recommendation.

In some embodiments, the treatment path may also identify one or more properties of the selected drug.

In some embodiments, the one or more properties of the selected drug may include one or more of: osmolarity, pH, or whether the selected drug is a vesicant.

In some embodiments, the method includes identifying one or more references that support the one or more treatment considerations. The treatment path may also include the one or more references.

In some embodiments, one or more computer storage media may be provided and may store computer executable instructions which when executed implement a method for dynamically generating a treatment path for infiltration or extravasation. An application programming interface (API) server can receive an identifier of a drug that has extravasated or infiltrated. The API server can receive an identifier of a grade of the extravasation or infiltration of the drug. The API server can query one or more data structures to identify one or more entries that associate the combination of the drug and the grade with one or more treatment considerations. A treatment path for the infiltration or extravasation can be presented to the clinician via a user interface. The treatment path can include the one or more treatment considerations that are associated with the combination of the drug and the grade.

In some embodiments, a system is provided where the system includes an API server that exposes one or more APIs by which a clinician can request a treatment path for extravasation or infiltration, and storage that stores one or more data structures that associate combinations of drugs and grades of extravasation or infiltration with one or more treatment considerations. The API server can be configured to implement a method for dynamically generating a treatment path. The method can include: receiving a request for a treatment path for extravasation or infiltration, the request identifying a drug that has extravasated or infiltrated and a grade of the extravasation or infiltration; querying the storage to obtain one or more treatment considerations that are associated with the combination of the drug and the grade; and causing the treatment path to be presented to the clinician, the treatment path including the one or more treatment considerations.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

These drawings depict only example embodiments and should not be considered limiting of the scope of the disclosed embodiments.

FIG. 1 illustrates an example computing environment in which one or more embodiments may be implemented.

FIGS. 2A-2G provide examples of data structures that may be used in one or more embodiments;

FIGS. 3A-3C provide an example of how a treatment path for extravasation of infiltration may be dynamically generated in one or more embodiments; and

FIGS. 4A-4C provide examples of user interfaces that may be presented to a clinician to allow the clinician to receive a dynamically generated treatment path for extravasation or filtration based on a selected drug and grade in accordance with one or more embodiments.

### DETAILED DESCRIPTION

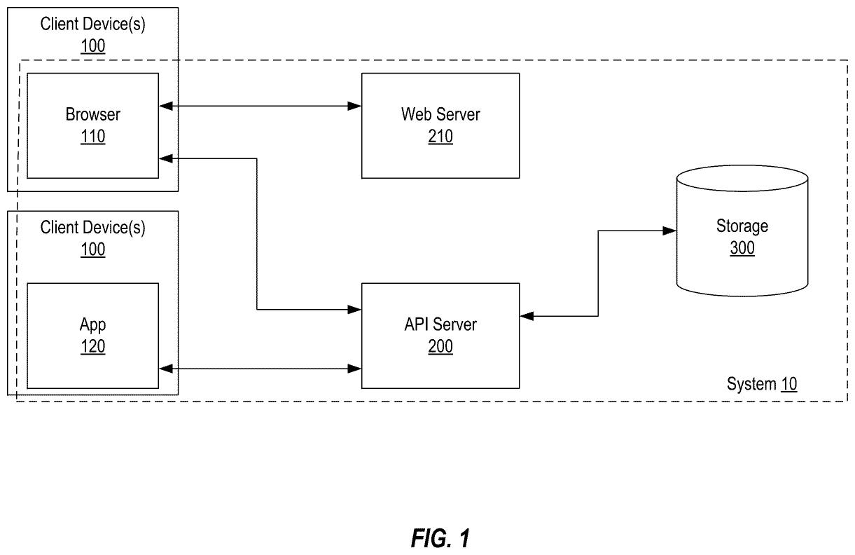

FIG. 1 provides an example of a computing environment in which embodiments of the present disclosure may be implemented. This environment includes a system 10 that may include an API server (or servers) 200, possibly a web server (or servers) 210, and storage 300. Users of client devices 100 may use a browser 110 to access web-based content provided by web server 210 and/or may use an app 120. In either case, the web-based content hosted in browser 110 or app 120 may interface with API server 200. Web-based content, app 120 or other software components on the client side will be referred to generally as client-side components. Of primary relevance to embodiments of the present disclosure, the client-side components are configured to enable a clinician to request, via API server 200, a treatment path for infiltration or extravasation. As described in detail below, a treatment path includes one or more treatment considerations that are selected based on the combination of a selected drug that infiltrated or extravasated and a selected grade of the infiltration or extravasation. A treatment path may also include one or more initial management recommendations, one or more post care management recommendations, and/or one or more additional recommendations (generally “recommendations”), all of which may be selected based on the combination of the selected drug and selected grade.

API server 200 can provide a number of APIs by which client devices 100 can interface with system 10 to implement functionality described herein. In this context, the term API should be construed broadly as encompassing an interface for receiving any type of communication for performing the functionality described herein. Likewise, the term API server should be construed as encompassing any service that is capable of performing the functionality described herein. In some embodiments, web server 210 and API server 200 may be the same server (or services on the same physical/virtual server).

Storage 300 can represent any type or number of storage mechanisms for storing the data structures that system 10 may use. For example, storage 300 may include relational databases, object storage, indexes, file systems, etc. Any or all of API server 200, web server 210, and storage 300 can be hosted in the cloud, implemented on dedicated hardware or provided in any other suitable manner.

Client device(s) 100 can represent any computing device that a clinician (or other user) may use to interface with system 10. For example, a client device 100 may be a desktop, a laptop, a tablet, a smart phone, a thin client, a vital signs monitor or other dedicated medical computing device, etc. In typical implementations, there may be many hospitals, medical offices, or other clinics that use system 10, and therefore, there may be many client devices 100 that clinicians associated with such locations use to access system 10. The term “clinician” should be construed as including any individual that may care for a patient having an IV line such as a nurse, doctor, pharmacist or licensed independent practitioner.

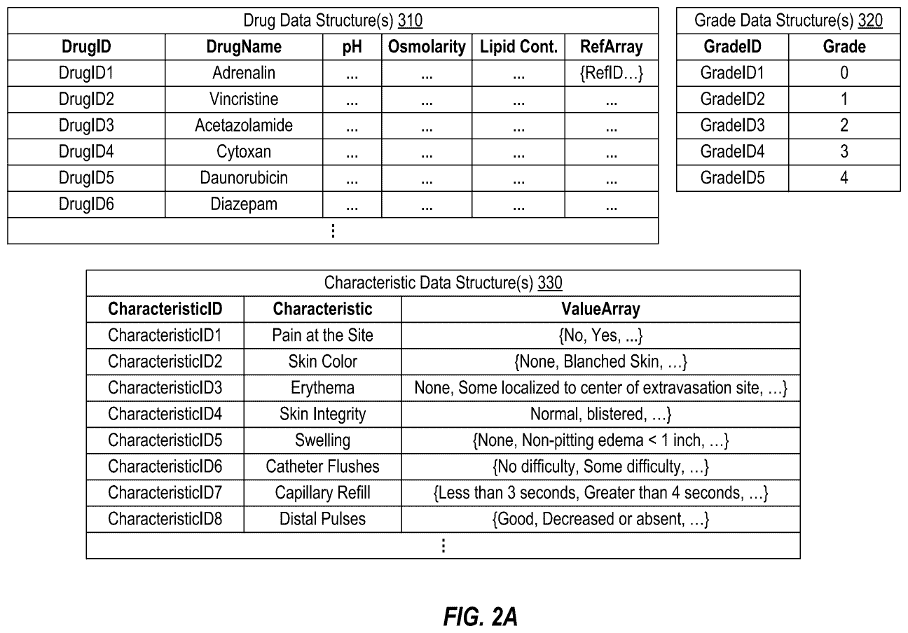

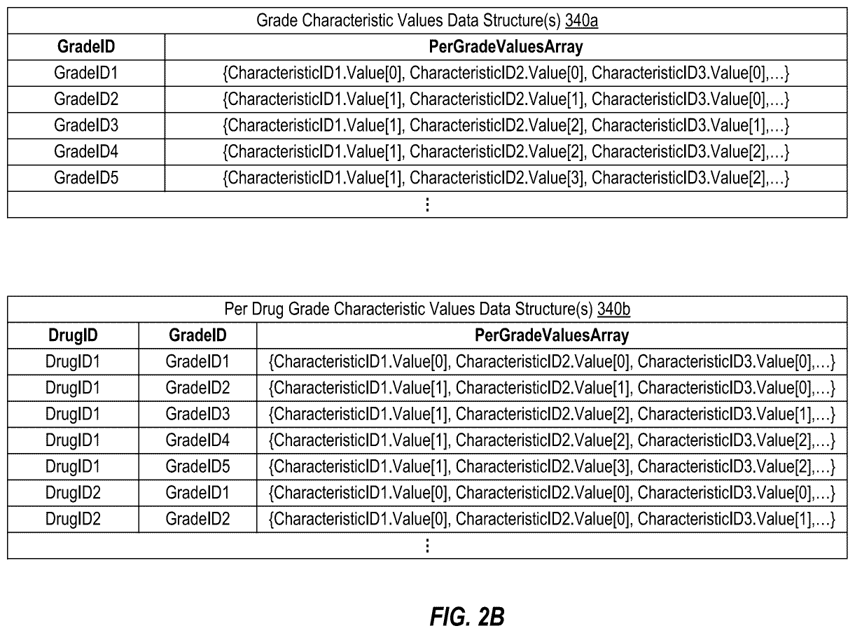

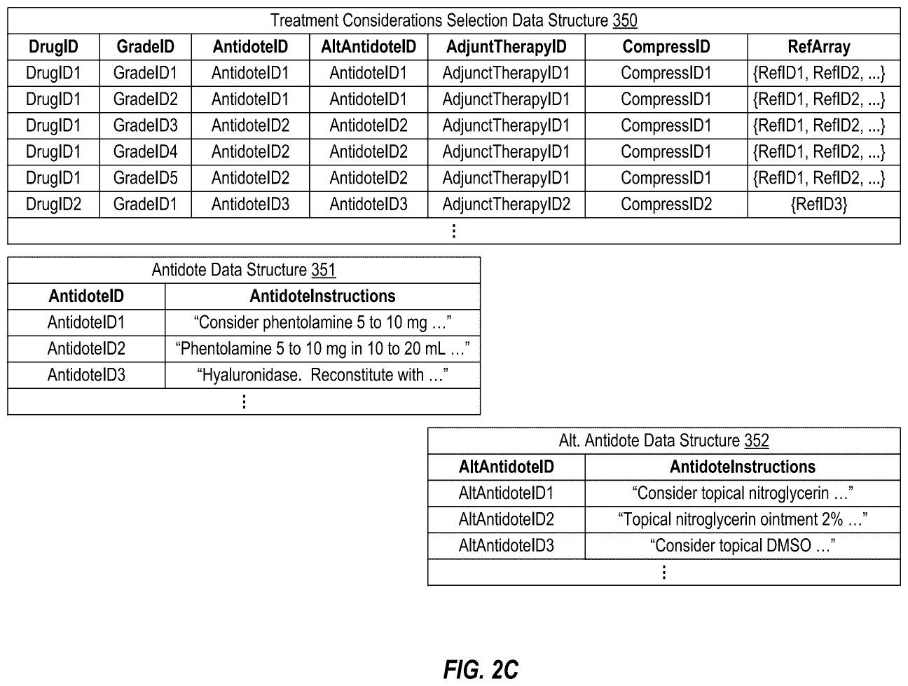

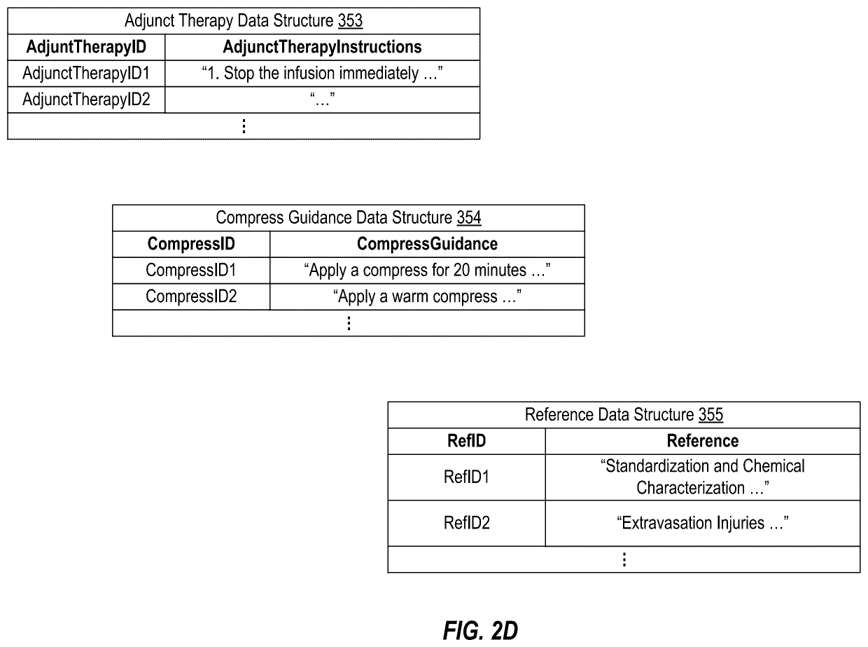

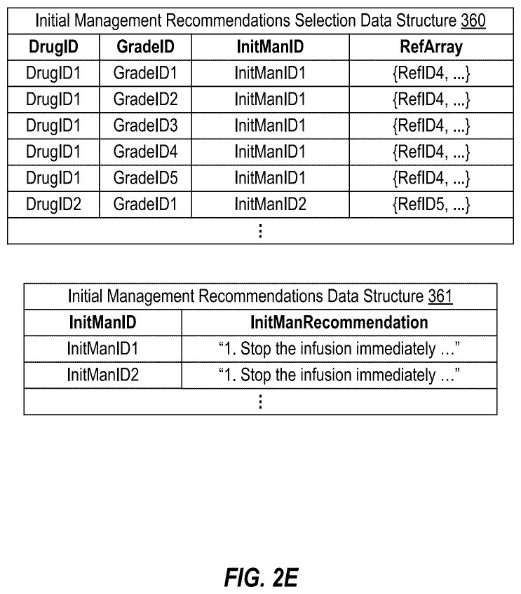

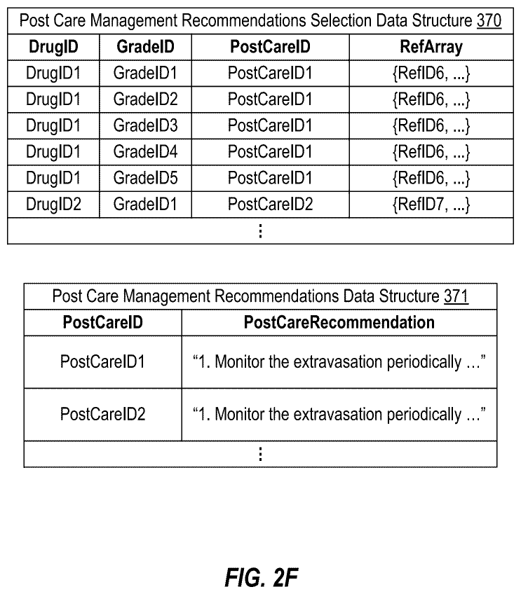

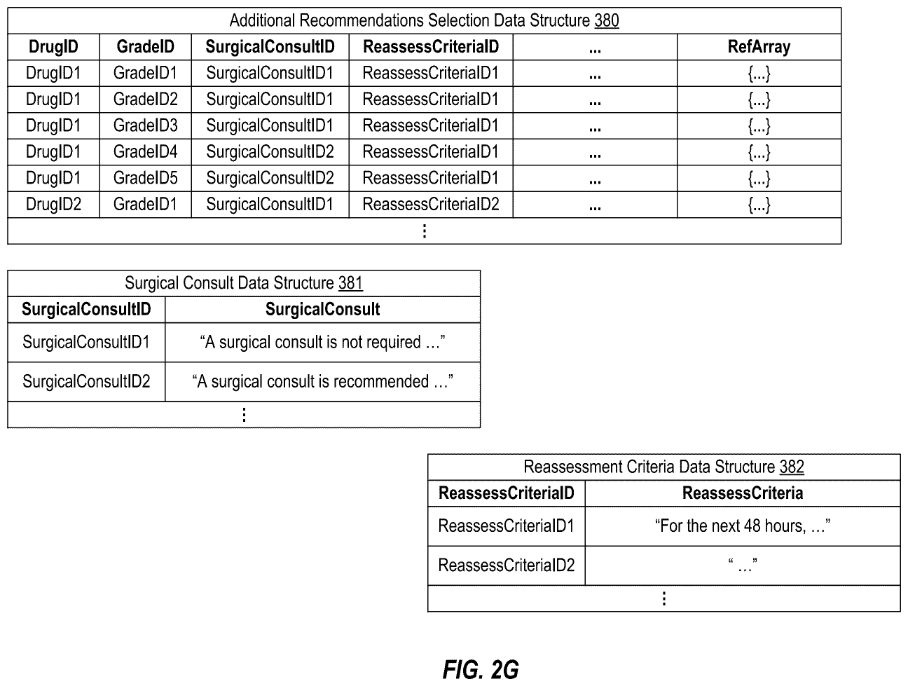

FIGS. 2A-2G provide examples of data structures that may be maintained in storage 300 and used by API server 200 to implement functionality described herein. The unique arrangement of the data in these data structures as well as how the APIs of API server 200 leverage this unique arrangement improves the functionality of system 10 for purposes of dynamically generating a treatment path for extravasation or infiltration.

FIG. 2A includes an example drug data structure (or structures) 310 which can be used to define each drug for which a treatment path may be provided when infiltration or extravasation occurs. For each drug, drug data structure 310 may associate the drug name with a unique identifier (DrugID) and with various characteristics of the drug such as its pH, its osmolarity, and its lipid content. In some embodiments, drug data structure 310 may also associate each drug with one or more references which may be the source(s) of the information defined in drug data structure 310 (and in other data structures as described below) and/or where information about the drug can be obtained and reviewed. In some embodiments, such references may be defined using a unique identifier (RefID).

FIG. 2A also includes an example grade data structure (or structures) 320 which can be used to define various grades that can be used to represent and determine the severity of an infiltration or extravasation. In the depicted example, there are five grades which are represented as numbers ranging from 0 to 4 with 0 being the least severe and 4 being the most severe. For each grade, grade data structure 320 may associate the grade with a unique identifier (GradeID).

FIG. 2A further includes a characteristic data structure (or structures) 330 which can be used to define various characteristics of an infiltration or extravasation along with various values for each characteristic. In the depicted example, there are eight characteristics which a clinician should consider when an infiltration or extravasation is suspected. For each characteristic, characteristic data structure 330 may associate the characteristic (or a description of the characteristic) with a unique identifier (CharacteristicID) and the possible values for the characteristic (e.g., in the form of an array (ValueArray)). These values can represent the severity of the corresponding characteristic or the degree to which the corresponding characteristic is present.

FIG. 2B includes two example variations of a characteristic values data structure (or structures) 340a and 340b (or generally 340). In general, characteristic values data structure 340 can be used to define for each grade a set of values for the characteristics that are most likely to be present when the severity of the infiltration or extravasation matches the corresponding grade. For example, characteristic values data structure 340 can define the values for the eight characteristics that are most likely to represent a grade 0 extravasation.

In a first example embodiment, grade characteristic values data structure 340a may be used to define the values of each characteristic for each grade. In such embodiments, these values may be the same regardless of the drug. In a second example embodiment, per drug grade characteristic values data structure 340b may be used to define the values of each characteristic for each grade and for each drug. In other words, in such embodiments, the values of the characteristics for each grade are dependent on the drug. In some embodiments, a hybrid between these characteristic values data structures 340a and 340b could be employed such that a first set of values for the characteristics for each grade may be shared among a first set of drugs, a second set of values for the characteristics for each grade may be shared among a second set of drugs, etc.

FIG. 2C provides an example of how system 10 may associate treatment considerations with a particular drug and grade of infiltration or extravasation. A treatment considerations selection data structure (or structures) 350 can associate a DrugID and GradeID pair with zero or more antidotes (e.g., using an AntidoteID), zero or more alternate antidotes (e.g., using an AltAntidoteID), zero or more adjunct therapies (e.g., using an AdjunctTherapyID), zero or more compression recommendations (e.g., using a CompressID), and zero or more references (e.g., using a RefID). Accordingly, by selecting a drug and a grade, the clinician can receive treatment considerations for the extravasation or infiltration.

FIGS. 2C and 2D provide examples of an antidote selection data structure (or structures) 351, an alternate antidote data structure (or structures) 352, an adjunct therapy data structure (or structures) 353, a compress guidance data structure (or structures) 354, and a reference data structure (or structures) 355 which may be used to define antidotes, alternate antidotes, adjunct therapies, compression guidance and references respectively.

Antidote data structure 351 associates each AntidoteID with the instructions for the corresponding antidote. For example, the antidote having AntidoteID1 as its AntidoteID may be phentolamine and may be associated with instructions of “Consider phentolamine 5 to 10 mg.” Similarly, AntidoteID2 may also represent phentolamine but may be associated with instructions of “Phentolamine 5 to 10 mg in 10 to 20 mL.” Accordingly, different instructions for treating a patient with the same antidote may be defined where the selection of particular instructions may be dependent on the drug and grade. Alternate antidote data structure 352 is similarly configured. As described further below, system 10 may select both an antidote and an alternate antidote for inclusion in a treatment path in some embodiments. Adjunct therapy data structure 353 and compress guidance data structure 354 are also structured in a similar manner but are used to associate an adjunct therapy and compression guidance with a particular drug and grade of infiltration or extravasation.

Reference data structure 355 may associate each RefID with the textual content (and possibly a hyperlink) for the reference. Such references can include support for and/or additional information about the selection of the treatment considerations in the treatment path for the particular drug and grade. Reference data structure 355 can be used in a similar manner for some or all of the recommendations that may be included in the treatment path.

FIG. 2E provides examples of an initial management recommendations selection data structure 360 and an initial management recommendations data structure 361 which, like the data structures described above, are used to associate zero or more initial management recommendations with a drug and grade and may also associate one or more references with such initial management recommendations.

FIG. 2F provides examples of a post care management recommendations selection data structure 370 and a post care management recommendations data structure 371 which, like the data structures described above, are used to associate zero or more post care management recommendations with a drug and grade and may also associate one or more references with such post care management recommendations.

FIG. 2G provides examples of an additional recommendations selection data structure 380, a surgical consult data structure 381, and a reassessment criteria data structure 382 (among possibly other data structures) which, like the data structures described above, are used to associate zero or more additional recommendations with a drug and grade and may also associate one or more references with such additional recommendations.

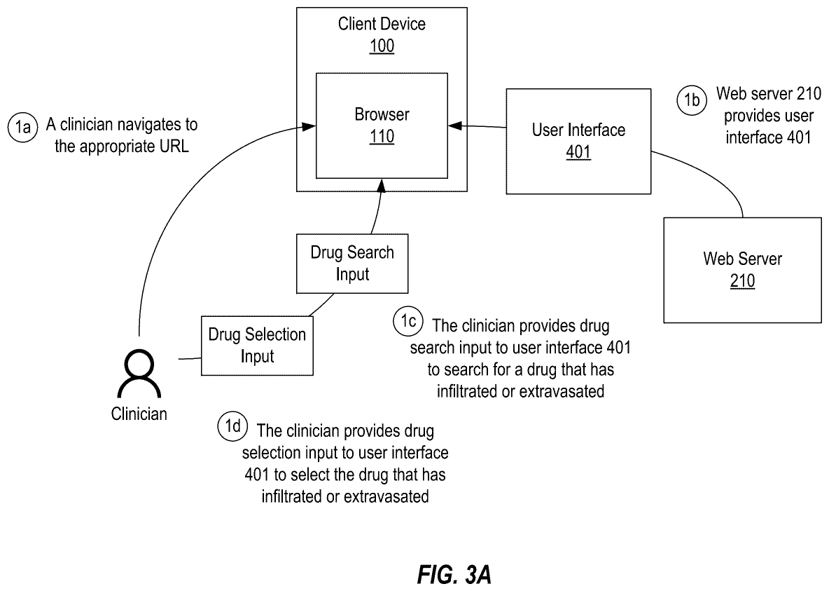

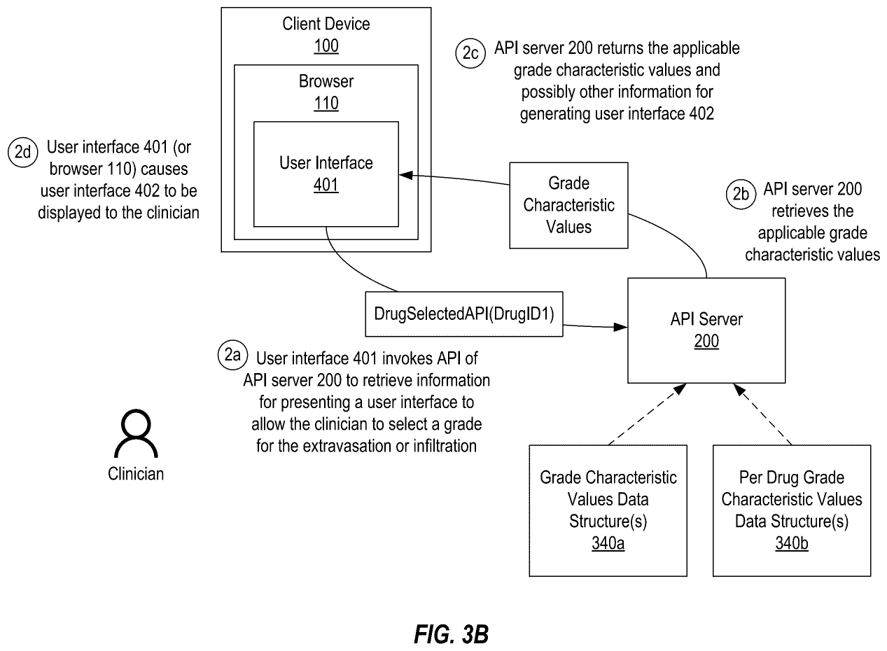

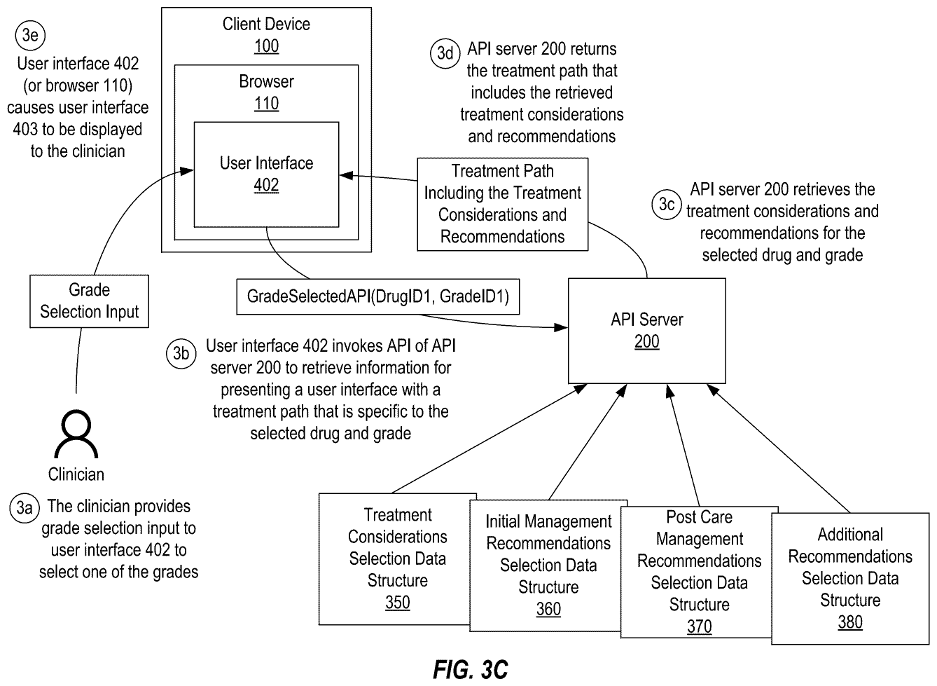

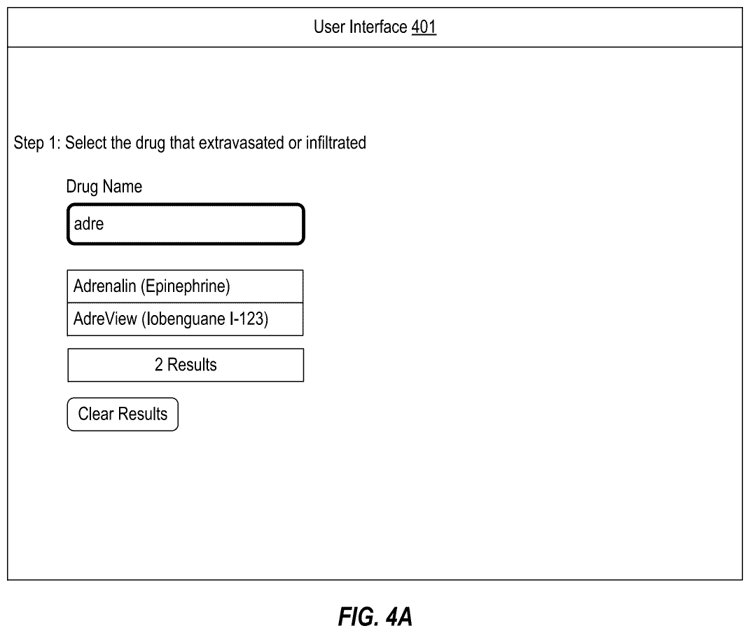

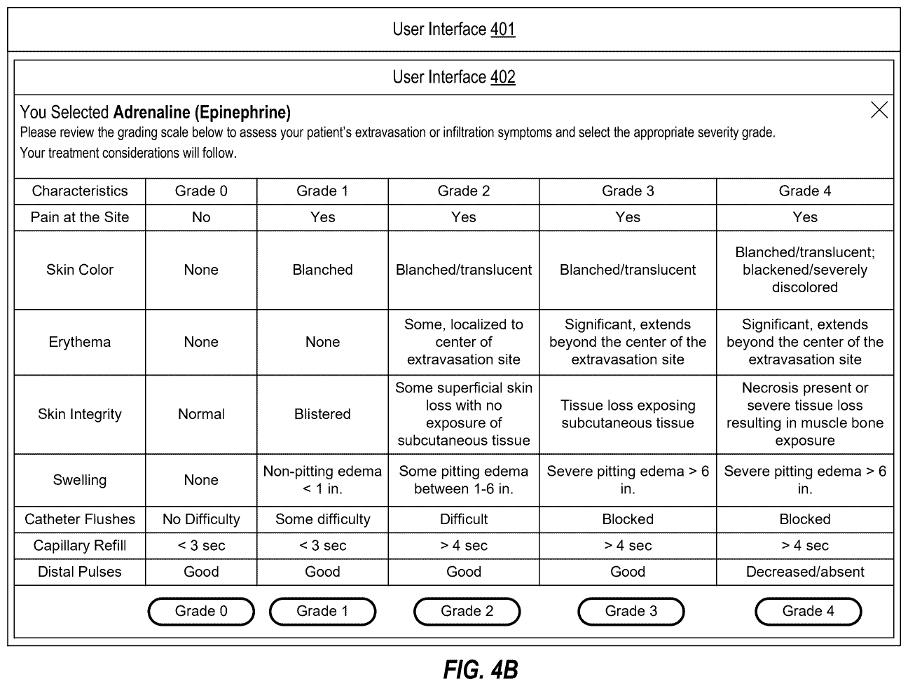

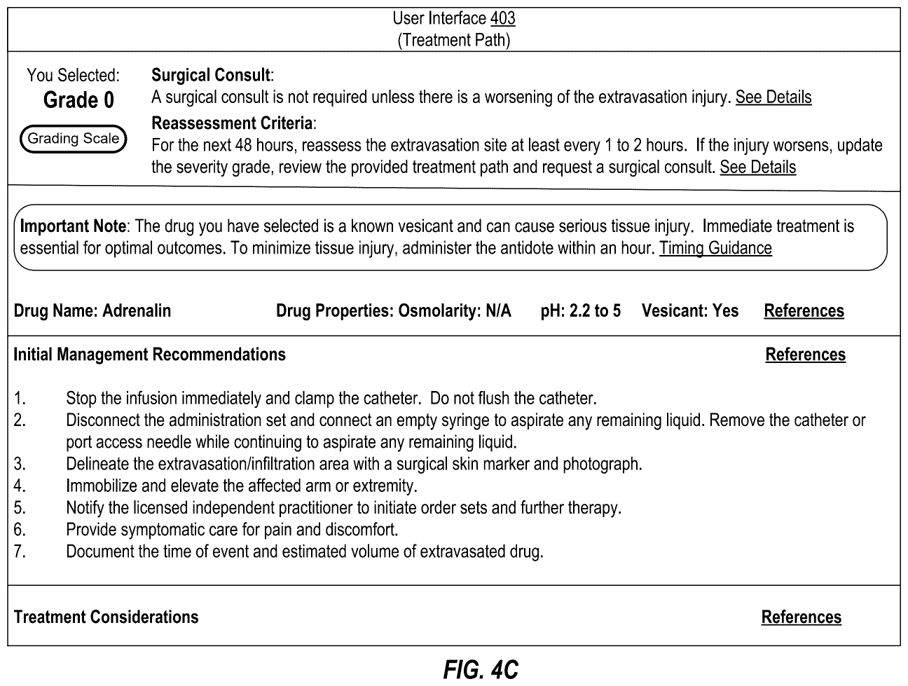

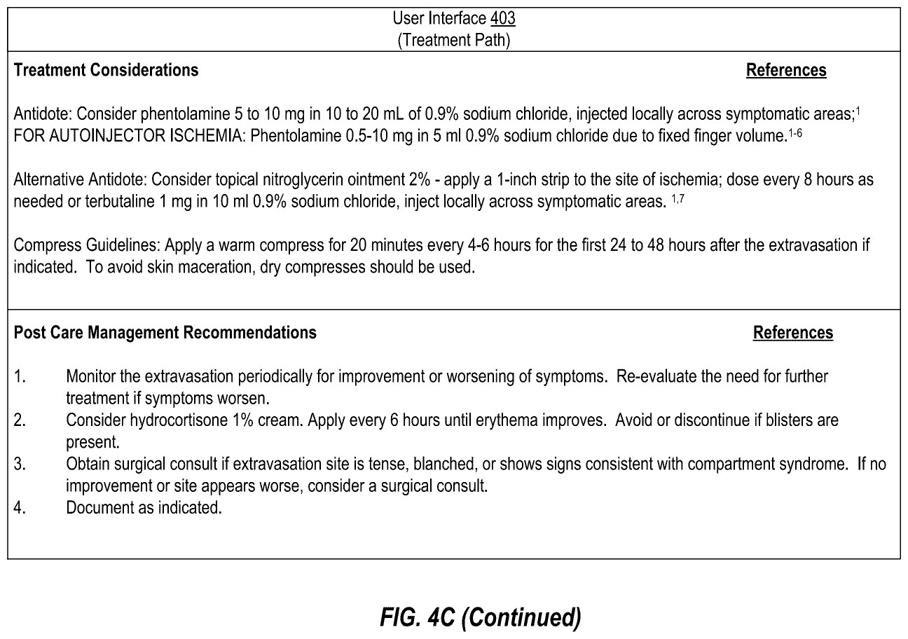

FIGS. 3A-3C provide an example of how system 10 can dynamically generate a treatment path for extravasation or infiltration. More particularly, these figures show how the unique arrangement of data and the unique configuration of API server 200 enable system 10 to more quickly, efficiently and accurately determine and present a treatment path that a clinician may follow. FIGS. 4A-4C are corresponding example user interfaces that may be presented to the clinician as the functionality represented in FIGS. 3A-3C is implemented. In this example, it is assumed that the clinician uses browser 110, but substantially the same functionality would be performed when the clinician uses app 120.

Turning to FIGS. 3A and 4A, in step la it is assumed that a clinician has detected extravasation or infiltration and has therefore used client device 100 to navigate browser 110 to the appropriate URL to thereby cause web server 210 to provide user interface 401 in step 1b. User interface 401 can instruct the clinician to select the drug that extravasated or infiltrated such as by providing a search box. In step 1c, the clinician can provide drug search input to user interface 401. In this example, it is assumed that the drug is adrenaline and that the drug search input is “adre” which causes user interface 401 to present Adrenalin and Adre View as possible results. Although not represented in FIG. 3A, user interface 401 could be configured to populate these possible results by invoking an API of API server 200 to query drug data structure 310 for entries having a value for the DrugName parameter that matches the drug search input. In step 1d, the clinician provides drug selection input to select the drug that has extravasated or infiltrated. In this example, it is assumed that the clinician selects the displayed result for Adrenalin in user interface 401.

Turning to FIGS. 3B and 4B, in step 2a, and in response to the clinician selecting the drug that has extravasated or infiltrated, user interface 401 may invoke an API of API server 200 to retrieve information for presenting a user interface by which the clinician can select a grade for the extravasation or infiltration. In some embodiments, the invocation of this API may include identifying the selected drug such as by specifying the DrugID. In step 2b, API server 200 can retrieve the appliable grade characteristic values. For example, when the grade characteristic values are the same for all drugs, API server 200 may access grade characteristic values data structure 340a to retrieve the grade characteristic values. In some embodiments, however, these grade characteristic values could be provided as part of user interface 401. In contrast, if the grade characteristic values are dependent on the selected drug, API server 200 may query per drug grade characteristic values data structure 340b to retrieve the grade characteristic values applicable to the selected drug. For example, API server 200 could use the DrugID passed in the API request of step 2a to identify each matching entry and to retrieve the values in the corresponding PerGrade ValuesArray. In step 2c, API server 200 can return the applicable grade characteristic values and possibly additional information that may be useful for assisting the clinician in selecting a grade (e.g., instructions that may be dependent on the selected drug). Finally, in step 2d, user interface 401 can be configured to cause a user interface 402 to be displayed to the clinician.

FIG. 4B provides an example of how user interface 402 could be configured. As shown, user interface 402 may be a popup window that identifies the selected drug and lists the characteristics the clinician should consider along with the values for these characteristics for each possible grade. User interface 402 can assist the clinician in quickly and accurately identifying the grade or severity of the extravasation of infiltration. For example, the clinician needs only identify which column most accurately identifies the characteristics that the patient is exhibiting. User interface 402 also includes selectable elements (buttons) for each grade. The clinician can select the appropriate element to select the grade for the extravasation or infiltration.

Turning to FIGS. 3C and 4C, in step 3a, the clinician may provide grade selection input to user interface 402 to select one of the grades for the extravasation or infiltration. For example, the clinician can review the characteristic values in user interface 402 and select the grade corresponding to the values that most closely match what the patient is exhibiting. In step 3b and in response to the grade selection input, user interface 402 can invoke an API of API server 200 to retrieve information for presenting a user interface with a treatment path that is specific to the selected drug and grade. For example, the API request could specify the DrugID and GradeID for the selected drug and grade. In step 3c, API server 200 may retrieve the treatment considerations and recommendations of the treatment path for the selected drug and grade. For example, API server 200 could use the DrugID and GradeID of the selected drug and grade to query treatment considerations selection data structure 350, initial management recommendations selection data structure 360, post care management recommendations selection data structure 370 and additional recommendations selection data structure 380 for matching entries and could then use such entries to gather the corresponding treatment consideration(s) (e.g., any antidote, alternate antidote, adjunct therapy, and compression guidance that may be associated with the selected drug and grade), the corresponding initial management recommendation(s), the corresponding post care management recommendation(s), and any corresponding additional recommendations, along with any associated references. In step 3d, API server 200 can return these treatment considerations and recommendations. Finally, in step 3e, user interface 402 (or browser 110) can cause a user interface 403 to be displayed to the clinician.

FIG. 4C provides an example of how user interface 403 could be configured to present the treatment path to the clinician. User interface 403 can include an indication of the selected grade and drug as well as relevant properties of the drug (e.g., the osmolarity, the pH, whether it is a vesicant, any references supporting these properties, etc., all of which could have been retrieved as part of step 2a. step 3b, or otherwise). In some embodiments, if the selected drug is a vesicant, user interface 403 may include an important note to inform the clinician that special precautions should be taken. In some embodiments, additional recommendations may also be presented in conjunction with the selected grade (e.g., whether a surgical consult is recommended, what the reassessment criteria are, etc.).

User interface 403 also includes the selected initial management recommendations, treatment considerations and post care management recommendations for the selected drug and grade (which are assumed to be Adrenalin and grade 0 in this example), along with supporting references. For example, the treatment considerations include an antidote, an alternative antidote, and compress guidelines.

Again, the treatment considerations and recommendations that make up a treatment path are dynamically selected based on the drug and grade the clinician selected. This dynamic selection enables the clinician to receive the proper guidance quickly without needing to consult many resources and/or individuals. Additionally, if the characteristics of the extravasation or infiltration change, the clinician can quickly receive an updated treatment path by simply selecting a different grade.

The arrangement of the data into the various data structures described above and the configuration of API server 200 to use these data structures improves the functioning of system 10. For example, existing systems rely on the storage of data in disparate systems and sources and do not provide a uniform mechanism for accessing the data. Furthermore, such existing systems, due to their configuration, are incapable of dynamically generating a treatment path for infiltration or extravasation based on the selection of a drug and grade. As a result, existing systems have failed to provide accurate and complete guidance to clinicians which in turn has resulted in unnecessary patient harm from infiltration and extravasation.

Embodiments of the present disclosure may comprise or utilize special purpose or general-purpose computers including computer hardware, such as, for example, one or more processors and system memory. Embodiments within the scope of the present disclosure also include physical and other computer-readable media for carrying or storing computer-executable instructions and/or data structures. Such computer-readable media can be any available media that can be accessed by a general purpose or special purpose computer system.

Computer-readable media are categorized into two disjoint categories: computer storage media and transmission media. Computer storage media (devices) include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other similarly storage medium which can be used to store desired program code means in the form of computer-executable instructions or data structures and which can be accessed by a general purpose or special purpose computer. Transmission media include signals and carrier waves. Because computer storage media and transmission media are disjoint categories, computer storage media does not include signals or carrier waves.

Computer-executable instructions comprise, for example, instructions and data which, when executed by a processor, cause a general-purpose computer, special purpose computer, or special purpose processing device to perform a certain function or group of functions. The computer executable instructions may be, for example, binaries, intermediate format instructions such as assembly language or P-Code, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAS, tablets, smart watches, pagers, routers, switches, and the like.

The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices. An example of a distributed system environment is a cloud of networked servers or server resources. Accordingly, the present disclosure can be hosted in a cloud environment.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description.

1. A method for dynamically generating a treatment path for an infiltration or extravasation that has already occurred, the method comprising:
after an infiltration or extravasation has already occurred, presenting, via a computing device, one or more user interfaces by which a clinician selects, from a plurality of drugs, a drug that has infiltrated or extravasated during administration to a patient and by which the clinician selects, from a plurality of grades, a grade of the infiltration or extravasation that has already occurred;
receiving, at an application programming interface (API) server, one or more API calls that identify the selected drug and the selected grade;
in response to the one or more API calls that identify the selected drug and the selected grade, querying, by the API server, one or more data structures to identify one or more entries that associate the combination of the selected drug and the selected grade with one or more treatment considerations; and
presenting, via the computing device, a treatment path for the infiltration or extravasation that has already occurred, the treatment path including the one or more treatment considerations that are associated with the combination of the selected drug and the selected grade such that the clinician receives the treatment path for the infiltration or extravasation that has already occurred by selecting the drug and grade.

2. The method of claim 1, wherein presenting the one or more interfaces by which the clinician selects the grade of the infiltration or extravasation that has already occurred comprises presenting a plurality of characteristics of infiltration or extravasation and a plurality of sets of values for the plurality of characteristics where each set of values corresponds with one of the plurality of grades.

3. The method of claim 2, wherein the sets of values for the plurality of characteristics are obtained in response to the clinician's selection of the drug.

4. The method of claim 2, wherein the plurality of characteristics are selected from a group comprising two or more of: pain at the site, skin color, erythema, skin integrity, swelling, catheter flushes, capillary refill, or distal pulses.

5. The method of claim 2, wherein the plurality of characteristics are selected from a group comprising all of: pain at the site, skin color, erythema, skin integrity, swelling, catheter flushes, capillary refill, and distal pulses.

6. The method of claim 1, wherein the plurality of grades include five grades.

7. The method of claim 1, further comprising:
querying, by the API server, the one or more data structures to identify one or more entries that associate the combination of the selected drug and the selected grade with one or more initial management recommendations;
wherein the treatment path for the infiltration or extravasation that has already occurred also includes the one or more initial management recommendations.

8. The method of claim 1, further comprising:
querying, by the API server, the one or more data structures to identify one or more entries that associate the combination of the selected drug and the selected grade with one or more post care management recommendations;
wherein the treatment path for the infiltration or extravasation that has already occurred also includes the one or more post care management recommendations.

9. The method of claim 1, further comprising:
querying, by the API server, the one or more data structures to identify one or more entries that associate the combination of the selected drug and the selected grade with one or more additional recommendations;
wherein the treatment path for the infiltration or extravasation that has already occurred also includes the one or more additional recommendations.

10. The method of claim 9, wherein the one or more additional recommendations include a surgical consult recommendation or a reassessment criteria recommendation.

11. The method of claim 1, wherein the treatment path for the infiltration or extravasation that has already occurred also identifies one or more properties of the selected drug.

12. The method of claim 11, wherein the one or more properties of the selected drug include one or more of: osmolarity, pH, or whether the selected drug is a vesicant.

13. The method of claim 1, further comprising:
identifying one or more references that support the one or more treatment considerations;
wherein the treatment path for the infiltration or extravasation that has already occurred also includes the one or more references.

14. One or more computer storage media storing computer executable instructions which when executed implement a method for dynamically generating a treatment path for an infiltration or extravasation that has already occurred, the method comprising:
after an infiltration or extravasation has already occurred, receiving, at an application programming interface (API) server, an identifier of a drug that has infiltrated or extravasated;
receiving, at the API server, an identifier of a grade of the infiltration or extravasation of the drug that has already occurred;
querying, by the API server, one or more data structures to identify one or more entries that associate the combination of the drug and the grade with one or more treatment considerations; and presenting, to a clinician and via a user interface, a treatment path for the infiltration or extravasation that has already occurred, the treatment path for the infiltration or extravasation that has already occurred including the one or more treatment considerations that are associated with the combination of the drug and the grade.

15. The computer storage media of claim 14, wherein the identifier of the grade of the infiltration or extravasation of the drug that has already occurred is received in response to presenting a plurality of characteristics of infiltration or extravasation and a plurality of sets of values for the plurality of characteristics where each set of values corresponds with one of the plurality of grades.

16. The computer storage media of claim 14, wherein the method further comprises:
querying, by the API server, the one or more data structures to identify one or more entries that associate the combination of the drug and the grade with one or more initial management recommendations;
wherein the treatment path for the infiltration or extravasation that has already occurred also includes the one or more initial management recommendations.

17. The computer storage media of claim 14, wherein the method further comprises:
identifying one or more references that support the one or more treatment considerations;
wherein the treatment path for the infiltration or extravasation that has already occurred also includes the one or more references.

18. A system comprising:
an API server that exposes one or more APIs by which a clinician can request a treatment path for an infiltration or extravasation that has already occurred; and
storage that stores one or more data structures that associate combinations of drugs and grades of infiltration or extravasation that has already occurred with one or more treatment considerations;
wherein the API server is configured to implement a method for dynamically generating a treatment path for an infiltration or extravasation that has already occurred, the method comprising:
receiving a request for a treatment path for infiltration or extravasation that has already occurred, the request identifying a drug that has infiltrated or extravasated and a grade of the infiltration or extravasation;
querying the storage to obtain one or more treatment considerations that are associated with the combination of the drug and the grade; and
causing the treatment path for the infiltration or extravasation that has already occurred to be presented to the clinician, the treatment path for the infiltration or extravasation that has already occurred including the one or more treatment considerations.

19. The system of claim 18, wherein the treatment path for the infiltration or extravasation that has already occurred also includes one or more initial management recommendations.

20. The system of claim 19, wherein the one or more treatment considerations also include one or more post care management recommendations.
